(19) United States
(12) Patent Application Publication
(10) Pub. No.: US 2004/0081580 A1
(43) Pub. Date: Apr. 29, 2004

(54) Use of nitric oxide and a device in the therapeutic management of pathogens in mammals

(76) Inventors: Doug Hole, Edmonton (CA); Christopher Miller, Vancouver (CA)

Correspondence Address: Kevin D. McCarthy, Roach Brown McCarthy & Gruber, P.C., 1620 Liberty Building, 420 Main Street, Buffalo, NY, 14202, US

(21) Appl. No.: 10/658,665

(22) Filed: Sep. 9, 2003

Related U.S. Application Data
(60) Provisional application No. 60/409,400, filed on Sep. 10, 2002.

(51) Int. Cl. A61M 1/14; A61M 37/00; C02F 1/44; A61M 1/36; A61M 1/34

(52) U.S. Cl. 422/44; 604/4.01; 604/23; 604/6.13; 210/645

(57) ABSTRACT

The present invention relates to a method for the systemic delivery of the nitric oxide moiety either as a dissolved gas or through the administration of nitric oxide donors in an extracorporeal circuit to reduce whole body bacterial contamination by pathogenic or toxic substrates. The utilization of an extracorporeal circuit with the entrainment of nitric oxide is viewed as a novel modality in the medical management of bacteremia (blood poisoning) and/or septicemia in mammals.

### CLAIM OF PRIORITY

This patent application claims priority to U.S. provisional patent application No. 60/409,400, filed on Sep. 9, 2002 and entitled “Use of extracorporeal gaseous nitric oxide, in the treatment of microbial septicemia and/or toxemia in mammals”.

### FIELD OF THE INVENTION

The present invention is directed to providing nitric oxide to mammals for medical applications.

### BACKGROUND OF THE PRESENT INVENTION

The present inventors have conducted a novelty search directed to their invention and determined that U.S. Pat. Nos. 6,432,077 to Stenzler and 5,957,880 to Igo are the most relevant references.

In the '880 reference, Igo taught that adding nitric oxide to blood within an extracorporeal system is known to inhibit platelet activation. Our summary of Igo's '880 reference is based on Igo's teaching which is as follows (bracketed material is added and underlining was added for emphasis):

Referring to FIG. 1, a typical CPB circuit is indicated generally by reference numeral 10. The patient is shown by numeral 12. A venous cannula 13 inserted into the patient is connected into a fluid inlet tube 14 that directs blood from the patient to a venous reservoir 18. Another cannula 15 inserted in the patient is connected to another fluid inlet 16 that also leads from the patient to venous reservoir 18. Reservoir 18 may be a pole mounted unit or may be located on the heart-lung machine table, but in either case normally is the first fixed point in the circuit, lines 14 and 16 normally being flexible and long enough to allow surgeon and surgical assistants room to maneuver around the surgical table. The purpose of venous reservoir 18 is to accumulate the admitted blood for feeding the balance of the CPB circuit. The accumulator eliminates pump starvation and cessation of pump prime by providing a buffer from ebb and flow of blood from the patient.

From the venous reservoir, plastic tubing 20 leads to the inlet side of a roller pump 22. Roller pump 22 has a hub 24 from which protrude two arms 26. These arms impinge on the tubing 20 collapsing it. Rotation of the pump hub 24 in the direction indicated by reference numeral 28 provides the desired flow direction and flow rate. The blood leaves the roller pump 22 through tubing 30 to the inlet of the oxygenator 32. The blood can be thermally adjusted by passing it from the oxygenator 32 through tubing 34 into a heat exchanger 36 for heating or cooling before returning to the oxygenator 32 by tubing 38. Upon oxygenation, the blood exists the oxygenator in two ways. The first way is through tubing 40 to another roller pump 42, from there pumped through tubing 44 to a cardioplegia system 46, then to the patient 12 through outlet tubing 47 and a cannula 48. The other mechanism with which the blood leaves the oxygenator 32 is through tubing 50. A filter 52 is located on a side branch of this portion of the circuit. When it is desired to use the filter 52, tubing 50 is clamped in the area noted by numeral 54 and the blood travels through the filter 52 before returning to the patient through outlet tubing 57 and a cannula 56. The venous return reservoir 18 is the juncture of all blood removed from the patient. It is at this location where the improvement according to this invention suitably may be added to the CPB circuit, prior to the pump 22 and the blood treatment oxygenator 32.

FIG. 2 depicts an extracorporeal blood treatment circuit in general, designated by reference numeral 11, and in which reference numerals are the same for the like elements found in the specific CPB circuit shown in FIG. 1. Reference numeral 41 represents a blood treatment component. In the case of a CPB apparatus as in FIG. 1, blood treatment component 41 comprises at least oxygenator 32 and optionally also heat exchanger 36 with connecting tubing 34, 38 and either or both of (1) the cardioplegia system 46 with associated second pump 42 and connecting tubing 40, 44, 47 and (2) the filter 52 with associated tubing 50. Numeral 17 indicates a blood fluid inlet generally and numeral 49 indicates a fluid outlet for blood return generally to the patient in FIG. 2. In accordance with this invention, blood treatment component 41 of the fluid circuit of the apparatus 11, instead of being an oxygenation system as in FIG. 1, suitably may be a heat exchange system 36, a renal dialysis component for exchange of urea and other blood chemicals with a dialysate solution across an exchange membrane, or an organ perfusion component such as an ex vivo liver and perfusion support system tying into circuit interconnects 30 and 49.

In accordance with this invention, one of more feeds of nitric oxide are employed, as necessary in the particular circuit, to maintain the concentration of nitric oxide in the circulating extracorporeal blood at a dosage effective to produce the desired inhibition of platelet activation over a period of time sufficient for the journey through the extracorporeal circulation apparatus yet insufficient to sustain the inhibition after the blood is returned to the patient and desired dosages. FIG. 3 depicts one such feed at the initial (venous inlet) portion of the circuit illustrated in FIG. 1. In this preferred embodiment of the invention, a gas permeable membrane 60 is located within a conduit 62 of the blood circuit located immediately downstream from the reservoir 18. The gas permeable membrane 60 is elongated and tubular in form and is disposed longitudinally within conduit 62 adapted to come into contact with blood flowing through conduit 62. A gaseous source, a mixture of nitric oxide and a carrier gas such as nitrogen, is housed in container 68 under high pressure. Regulator 66 controls the output gas pressure to periodic driver 69. The purpose of the periodic driver 69 is to induce a sinusoidal shaped pressure curve to the gas much like a “pulse”. The gas leaves the driver through tubing 64 and flows into the interior of gas permeable membrane 60. Due to the permeability of this membrane 60 to nitric oxide gas, the gas will diffuse through the membrane and dissolve in the blood plasma where it will come into contact with platelets. The membrane is selected to be impermeable to nitrogen and the nitrogen carrier gas will not diffuse through the membrane. Coupled to the outlet of the membrane 60 is outlet tubing 61, which is connected to valve 63. Valve 63 adjusts the back pressure of the system. From the valve 63 the carrier gas and any residual nitric oxide gas is carried through tube 65 into container 67, which is filled with a scavenger liquid such as methylene blue. The gas mixture is allowed to bubble up through the container containing the scavenger liquid. The scavenger liquid absorbs any residual nitric oxide so that the only gas that escapes into the atmosphere is the carrier gas.

Blood guarded by dissolved nitric oxide exits conduit 62 and into tubing 20 where is passes by a conventional blood flow measuring device 90. Signals from blood flow measuring device 90 are transferred by line 92 to controller feedback logic component 94 which outputs a signal through line 96 to controller driver component 98 for controlling pressure and flow from regulator 66. The controller system comprising units 90, 94 and 98 with connecting lines 92 and 96 controls the flow of gas into membrane 60 in relation to the flow of blood through tubing 20. In this manner, when the flow rate of the blood is low, the nitric oxide introduction is correspondingly and automatically reduced. Conversely, in cases of high flow the nitric oxide introduction is correspondingly and automatically raised.

The gas permeable membrane 62 has a gas permeable rate K which is dependent on the material of construction and the molecular characteristics of the gas. For nitric oxide, the gaseous release rate from membrane 60 is proportional to K, the exposed surface of the membrane to the blood, the internal gaseous pressure within the membrane and the hydraulic pressure of and gas tension of nitric oxide (if any) in the blood flowing by it. Delivered molecular concentrations to the blood is [sic] calculated knowing the above plus the absorption coefficient of the blood to the nitric oxide. Thus the controller controls the gas flow and at a level which, for the characteristics of membrane 60 and the absorption coefficient of nitric oxide gas at the temperature of the blood in the apparatus (before thermal adjustment, if any), is sufficient to provide an actual concentration of nitric oxide in solution effective in the presence of venous red blood cell blood hemoglobin to inhibit platelet activation.

FIG. 4 illustrates a longitudinal sectional view of the conduit 62, the gas permeable membrane 60 and the tubing 64. Nitric oxide gas flows into the membrane 60 at location 70. As the gas pressure inside the gas permeable membrane 60 exceeds the pressure of the blood within conduit 62, nitric oxide gas will diffuse from the membrane into the blood stream as indicated by arrows 74. The nitric oxide will be absorbed by the blood cellular components which will mediate the inflammatory response as described earlier.

Referring to FIG. 5, which illustrates a cross section of FIG. 4 along the line A—A, the relationship between the geometry's of the conduit 62 and gas permeable membrane 60 is as follows. The cross sectional area of the inside of conduit 62 minus the sectional area of the gas permeable membrane 60 (such difference being referenced by numeral 76) is approximately equivalent to the cross section of the tubing elsewhere in the CPB circuit, (i.e. the cross section of tubing element 20). With this relationship the blood is not subjected to an adverse pressure gradient in conduit 62. Longitudinally, the shape of the gas permeable membrane 60 follows that of the conduit 62, again so that adverse pressure gradients are not imparted into the circuit.

FIG. 6 illustrates another preferred embodiment of the invention. In this embodiment a carrier gas is not used so that container 68 holds a 100% concentration of nitric oxide. A pulse drive generator 69 is not shown but may be present. In this embodiment, there is no outlet conduit of membrane 60. As pressure builds up in conduit 60, the nitric oxide diffuses into the bloodstream as previously described. Because there are no residual carrier gas molecules, there is no need for a return. Simply stated, components 61, 63, 65, and 67 of the embodiment depicted in FIG. 2 are absent at the distal end of membrane 60 and the tube 62 in this configuration. As in the embodiment depicted in FIG. 3, a controller comprising components 90, 94 and 98 with connections 92 and 96 controls the concentration of nitric oxide in solution in the blood. FIG. 8 illustrates a cross sectional view B—B of FIG. 7 with the same numbers used in the same way as in FIG. 5.

The above embodiments illustrate an optimal configuration of the invention in which the blood flows around the external portion of a gas permeable membrane 60. While it is within the scope of this invention that the system can be configured so that the gas is on the external portion of the membrane and blood is flowed within the membrane, in low gas pressure conditions some membranes dilate, increasing the cross sectional area of the membrane and lowering blood flow through that portion of the apparatus, and in high gas pressure conditions, some membranes might collapse, reducing blood flow. In the preferred embodiments, if gas flow is zero, the membrane might collapse but it would not occlude or preclude blood flow.

FIG. 9 depicts another embodiment of the [Igo] invention. In this embodiment the nitric oxide feed is to reservoir 18. The feed comprises a diffuser 100 for diffusing nitric oxide gas into the reservoir, and comprises a regulator 66 for controlling gas pressure and rate of flow into the reservoir and a driver 69 for delivering the nitric oxide gas into reservoir 18 through inlet 64 in a pulsatile manner. Suitably diffuser 100 comprises a membrane or filter 80 that is not permeable to blood and is permeable to nitric oxide gas through which nitric oxide gas is introduced into the reservoir. As in the embodiment depicted in FIGS. 3 and 6, a controller comprising components 90, 94 and 98 with connections 92 and 96 controls the concentration of nitric oxide in solution in the blood.

It is important that the location of the nitric oxide feed be close to the patient cannulation point as possible in the extracorporeal circuit to reduce so much as practicable the period of exposure of platelets to non-endothelial surfaces. At least one feed location is described generally as upstream of the pump that is needed to circulate the blood extracorporeally through the system and back to the patient. With reference to the FIG. 2, that point is anywhere in line [14]. In FIGS. 3-9, which involve a CPB circuit where blood from two inlets 14 and 16 is pooled in reservoir 18, either the reservoir or the tubing immediately past the reservoir is selected for initial introduction of the nitric oxide, for the practical reason that these are the closest stationary locations in the system to the patient source of blood and also because control of nitric oxide introduction is most readily accomplished in the reservoir or in the blood filled lines in the immediately downstream tubing under the influence of a pump as opposed to in the blood inlet lines where lines are mobile to allow access to the surgical field, and especially in the case of blood suctioned from the operative field where intermittent blood and air flow occurs. The closest stationary location will vary according to the blood treatment component 41 involved in the use of this invention. Because of the very short half life of nitric oxide in the blood, additional feeds may be used further downstream to maintain the desired nitric oxide concentration in the blood without overdosing the blood in but one location.

In other words, Igo teaches away from adding nitric oxide to blood to combat pathogens.

In that '077 reference, Stenzler teaches that topical application of nitric oxide to wounds and/or skin of mammals is beneficial to wound healing because it decreases further infection. No where does Stenzler teach, disclose or suggest exposing nitric oxide to blood to combat pathogens. Our summary of Stenzler is based on his disclosure, which reads as follows:

The treatment of infected surface or subsurface lesions in patients has typically involved the topical or systemic administration of anti-infective agents to a patient. Antibiotics are one such class of anti-infective agents that are commonly used to treat an infected abscess, lesion, wound, or the like. Unfortunately, an increasingly number of infective agents such as bacteria have become resistant to conventional antibiotic therapy. Indeed, the increased use of antibiotics by the medical community has led to a commensurate increase in resistant strains of bacteria that do not respond to traditional or even newly developed anti-bacterial agents. Even when new anti-infective agents are developed, these agents are extremely expensive and available only to a limited patient population.

Another problem with conventional anti-infective agents is that some patients are allergic to the very compounds necessary to their treat their infection. For these patients, only few drugs might be available to treat the infection. If the patient is infected with a strain of bacteria that does not respond well to substitute therapies, the patient's life can be in danger.

A separate problem related to conventional treatment of surface or subsurface infections is that the infective agent interferes with the circulation of blood within the infected region. It is sometimes the case that the infective agent causes constriction of the capillaries or other small blood vessels in the infected region which reduces bloodflow. When bloodflow is reduced, a lower level of anti-infective agent can be delivered to the infected region. In addition, the infection can take a much longer time to heal when bloodflow is restricted to the infected area. This increases the total amount of drug that must be administered to the patient, thereby increasing the cost of using such drugs. Topical agents may sometimes be applied over the infected region. However, topical anti-infective agents do not penetrate deep within the skin where a significant portion of the bacteria often reside. Topical treatments of anti-infective agents are often less effective at eliminating infection than systemic administration (i.e., oral administration) of an anti-infective pharmaceutical.

In the 1980's, it was discovered by researchers that the endothelium tissue of the human body produced nitric oxide (NO), and that NO is an endogenous vasodilator, namely, and agent that widens the internal diameter of blood vessels. NO is most commonly known as an environmental pollutant that is produced as a byproduct of combustion. At high concentrations, NO is toxic to humans. At low concentrations, researchers have discovered that inhaled NO can be used to treat various pulmonary diseases in patients. For example, NO has been investigated for the treatment of patients with increased airway resistance as a result of emphysema, chronic bronchitis, asthma, adult respiratory distress syndrome (ARDS), and chronic obstructive pulmonary disease (COPD).

NO has also been investigated for its use as a sterilizing agent. It has been discovered that NO will interfere with or kill the growth of bacteria grown in vitro. PCT International Application No. PCT/CA99/01123 published Jun. 2, 2000 discloses a method and apparatus for the treatment of respiratory infections by NO inhalation. NO has been found to have either an inhibitory and/or a cidal effect on pathogenic cells.

While NO has shown promise with respect to certain medical applications, delivery methods and devices must cope with certain problems inherent with gaseous NO delivery. First, exposure to high concentrations of NO is toxic, especially exposure to NO in concentrations over 1000 ppm. Even lower levels of NO, however, can be harmful if the time of exposure is relatively high. For example, the Occupational Safety and Health Administration (OSHA) has set exposure limits for NO in the workplace at 25 ppm time-weighted averaged for eight (8) hours. It is extremely important that any device or system for delivering NO include features that prevent the leaking of NO into the surrounding environment. If the device is used within a closed space, such as a hospital room or at home, dangerously high levels of NO can build up in a short period of time.

Another problem with the delivery of NO is that NO rapidly oxidizes in the presence of oxygen to form NO₂, which is highly toxic, even at low levels. If the delivery device contains a leak, unacceptably high levels NO₂ of can develop. In addition, to the extent that NO oxides to form NO₂, there is less NO available for the desired therapeutic effect. The rate of oxidation of NO to NO₂ is dependent on numerous factors, including the concentration of NO, the concentration of O₂, and the time available for reaction. Since NO will react with the oxygen in the air to convert to NO₂, it is desirable to have minimal contact between the NO gas and the outside environment.

Accordingly, there is a need for a device and method for the treatment of surface and subsurface infections by the topical application of NO. The device is preferably leak proof to the largest extent possible to avoid a dangerous build up of NO and NO₂ concentrations. In addition, the device should deliver NO to the infected region of the patient without allowing the introduction of air that would otherwise react with NO to produce NO₂. The application of NO to the infected region preferably decreases the time required to heal the infected area by reducing pathogen levels. The device preferably includes a NO and NO₂ absorber or scrubber that will remove or chemically alter NO and NO₂ prior to discharge of the air from the delivery device.

In a first aspect of the [Stenzler] invention, a device for the topical delivery of nitric oxide gas to an infected area of skin includes a source of nitric oxide gas, a bathing unit, a flow control valve, and a vacuum unit. The bathing unit is in fluid communication with the source of nitric oxide gas and is adapted for surrounding the area of infected skin and forming a substantially air-tight seal with the skin surface. The flow control valve is positioned downstream of the source of nitric oxide and upstream of the bathing unit for controlling the amount of nitric oxide gas that is delivered to the bathing unit. The vacuum unit is positioned downstream of the bathing unit for withdrawing gas from the bathing unit.

In a second aspect of the [Stenzler] invention, the device according to the first aspect of the invention includes a controller for controlling the operation of the flow control valve and the vacuum unit.

In a third aspect of the [Stenzler] invention, the device according to the first aspect of the invention further includes a source of dilutent gas and a gas blender. The dilutent gas and the nitric oxide gas are mixed by the gas blender. The device also includes a nitric oxide gas absorber unit that is positioned upstream of the vacuum unit. The device also includes a controller for controlling the operation of the flow control valve and the vacuum unit.

In a fourth aspect of the [Stenzler] invention, a method of delivering an effective amount of nitric oxide to an infected area of skin includes the steps of providing a bathing unit around the infected area of skin, the bathing unit forming a substantially air-tight seal with the skin. Gas containing nitric oxide is then transported to the bathing unit so as to bathe the infected area of skin with gaseous nitric oxide. Finally, at least a portion of the nitric oxide gas is evacuated from the bathing unit.

It is an object of the [Stenzler] invention to provide a delivery device for the topical delivery of a NO-containing gas to an infected area of skin. It is a further object of the device to prevent the NO-containing gas from leaking from the delivery device. The method of delivering an effective amount of nitric oxide gas to the infected area of skin kills bacteria and other pathogens and promotes the healing process.

As clearly illustrated, Stenzler never taught, suggested, nor disclosed exposing blood to NO to destroy pathogens.

In 1989 it was discovered that nitric oxide was produced by the endothelium tissue of mammals. It has since been demonstrated that endogenous nitric oxide is a potent modulator for a number of systemic functions in mammals including selective pulmonary vasodilatation, neurotransmission and cytoxic activity over a wide range of microorganisms including bacteria and viruses. Nitric oxide has been known for years as an environmental pollutant and is toxic to mammals at high doses. At minimal concentrations however exogenously supplied (eg. <100 ppm) nitric oxide has selectively been used to treat human patients with a wide range of pulmonary diseases including, but not limited to, chronic bronchitis, asthma, ARDS (Acute Respiratory Disease Syndrome) etc. Nitric oxide has also found utility in its application as both a sterilizing agent and as a bactericidal agent for pathogenic organisms

Septicemia is a serious, rapidly progressive, life-threatening infection that can arise from infections throughout the body, including infections in the lungs, abdomen, and urinary tract. It may precede or coincide with infections of the bone (osteomyelitis), central nervous system (meningitis), or other tissues. Septicemia can rapidly lead to septic shock and death. Septicemia associated with some organisms such as meningococci can lead to shock, adrenal collapse and disseminated intravascular coagulopathy.

In all examples referenced there is a dosage range of nitric oxide application that needs to be maintained in order to establish efficacy. Accordingly the employment of nitric oxide as a dissolved gas or through selective nitric oxide donors in an extracorporeal circuit allows for the titration of exogenously administered nitric oxide levels required to optimize the therapeutic antimicrobial and bactericidal benefits.

The present invention introduces the concept of utilization and/or methods of application of gaseous nitric oxide (or via nitric oxide donors) in the treatment of blood to reduce pathogenic and/or toxic substrates in mammals. The prevention of bacteremia/septicemia in patients via management of extracorporeal blood, for example, by dialysis, perfusion, heat exchange or oxygenation relates to the methods and means sited to reduce the incidence of whole body infection in those patients.

### SUMMARY OF THE PRESENT INVENTION

The present invention relates to a method for the systemic delivery of the nitric oxide moiety either as a dissolved gas or through the administration of nitric oxide donors in an extracorporeal circuit or to a patient to reduce whole body bacterial contamination by pathogenic or toxic substrates. The utilization of an extracorporeal circuit and the patient's intake with the entrainment of nitric oxide to blood is viewed as a novel modality in the medical management of bacteremia (blood poisoning) and/or septicemia in mammals.

### BRIEF DESCRIPTION OF THE DRAWINGS

FIGS. 1-8 are prior art.

FIG. 9 is a schematic of the present invention.

FIG. 10 is an alternative embodiment of the present invention.

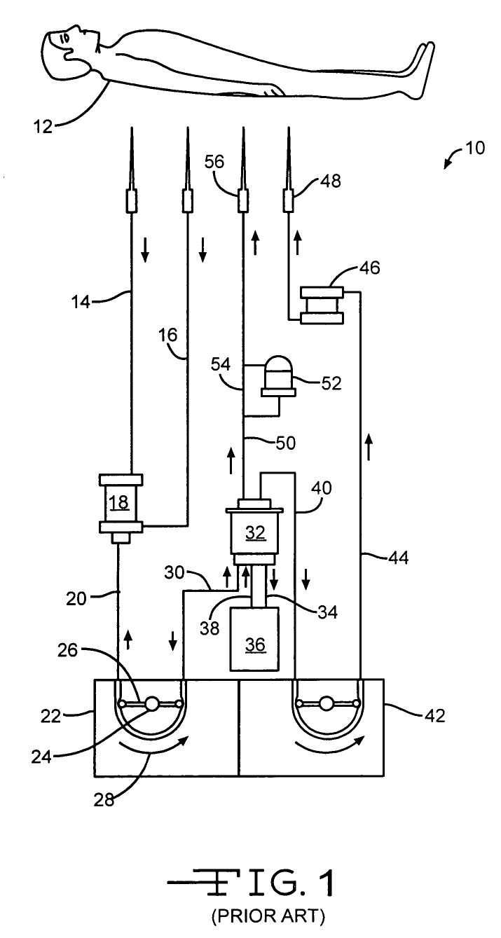

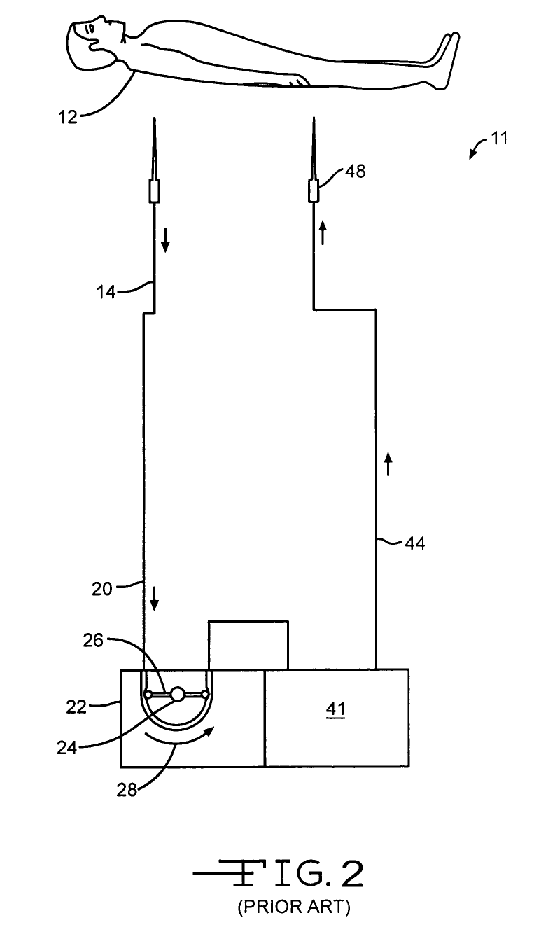

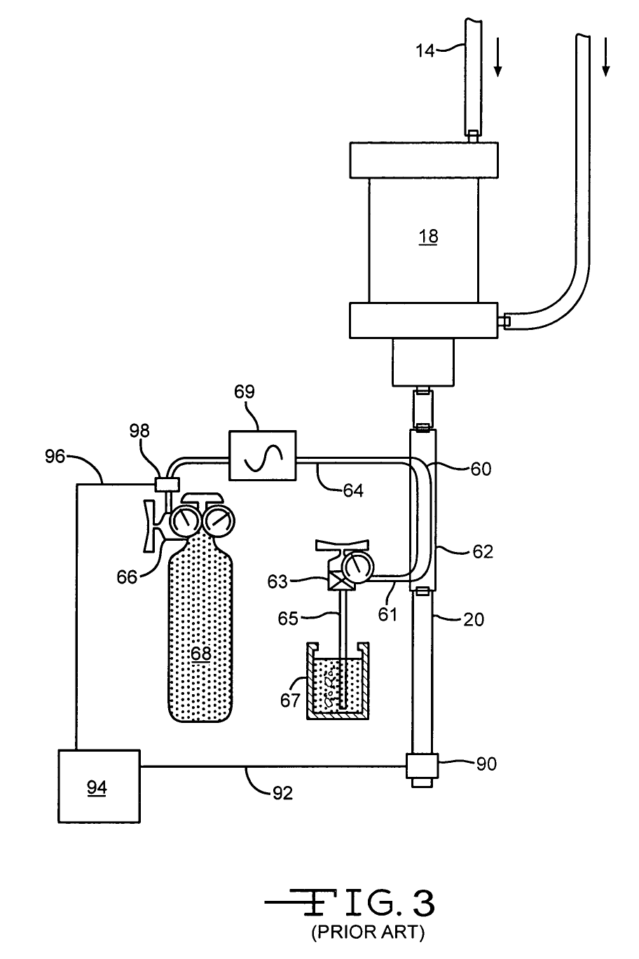

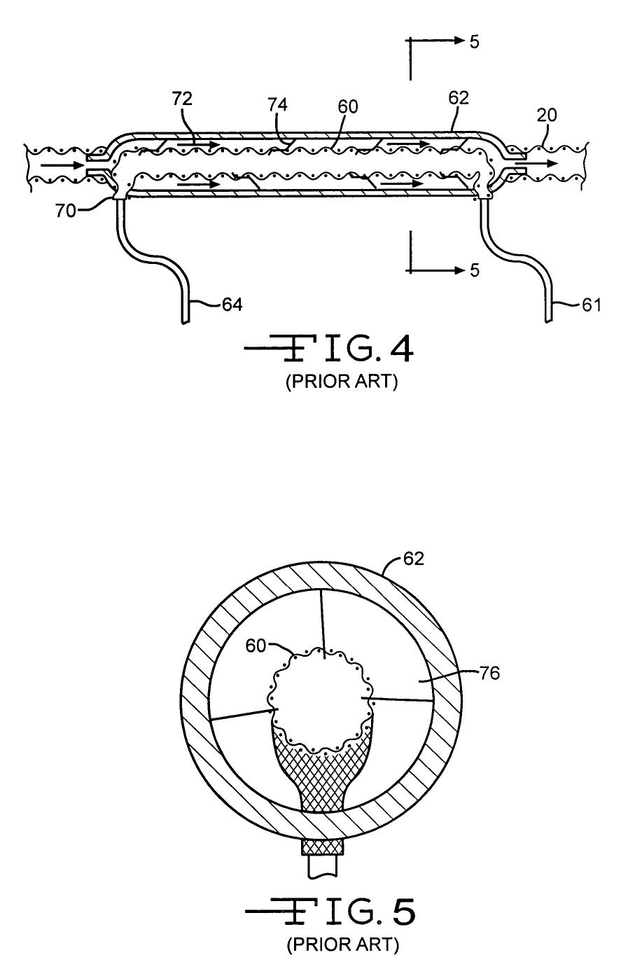

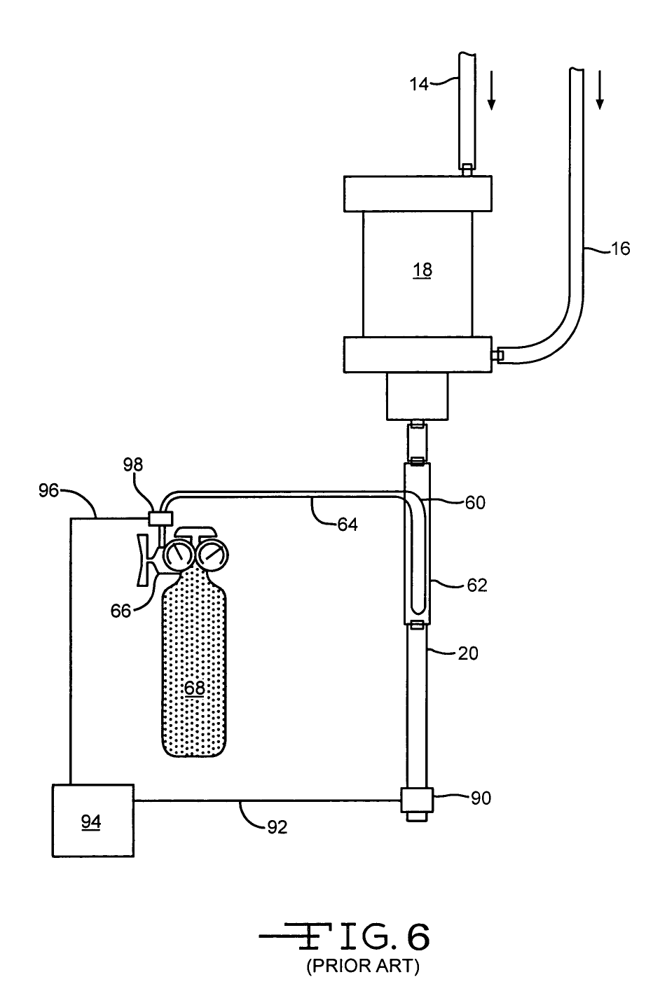

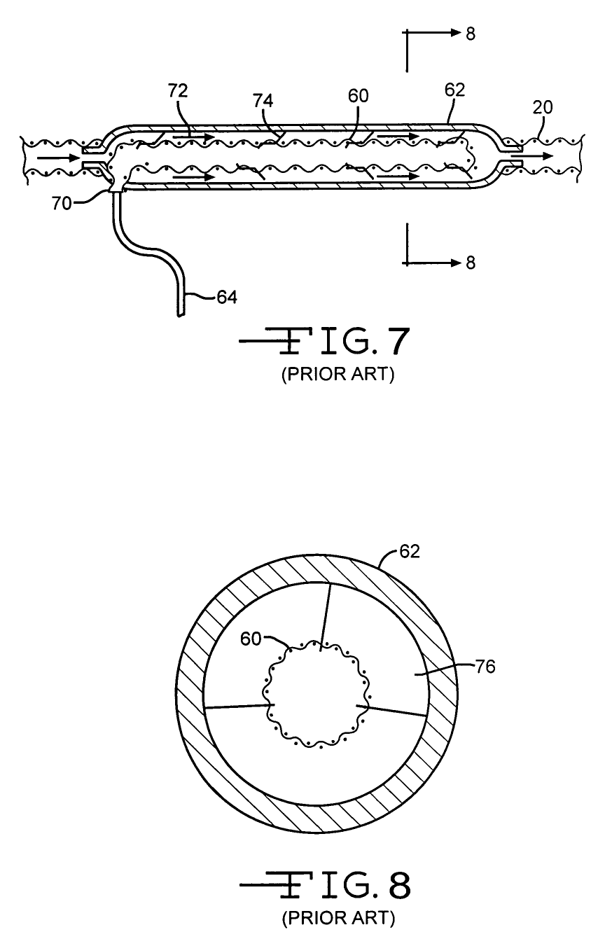

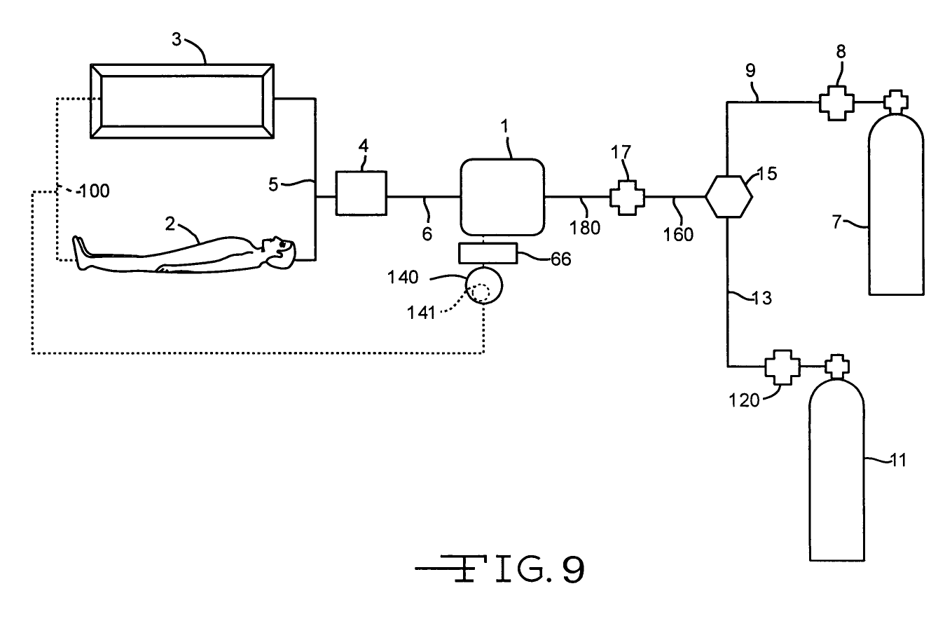

### DETAILED DESCRIPTION OF PREFERRED EMBODIMENTS

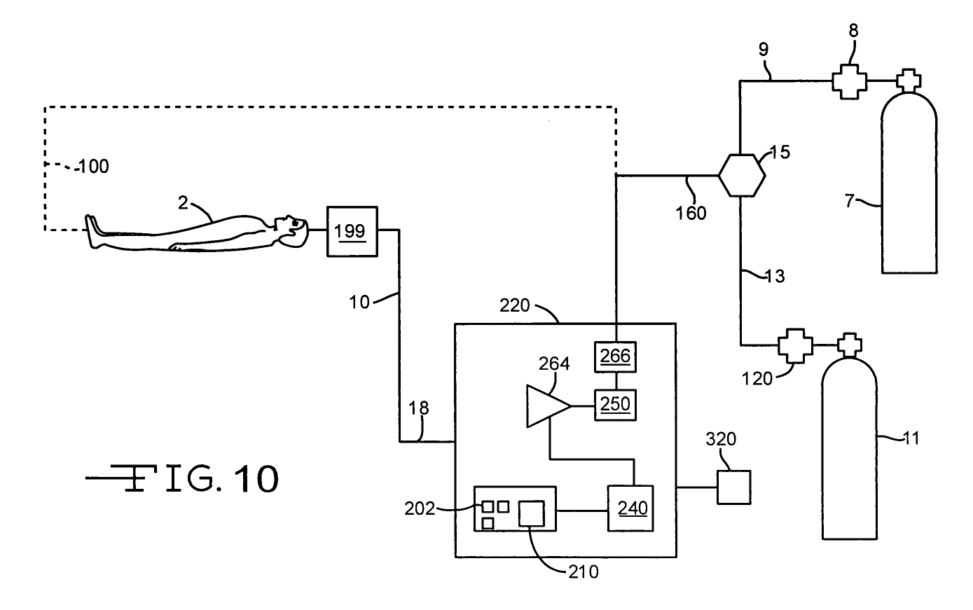

Referring now to FIG. 9, a gaseous nitric oxide (NO) delivery device 1 is shown connected to a source of infected blood from either a patient 2 or a stored blood source 3, and a pumping system 4, through lines 5, 6.

The nitric oxide (NO) source 7, can be a pressurized cylinder containing nitric oxide (NO) gas, and a nitric oxide flow control valve/pressure regulator 8, delivering nitric oxide (NO) to the gaseous nitric oxide delivery device 1 through supply tubing 9 and an optional gas blender 15. The infected blood is then exposed to a controlled amount of nitric oxide (NO) by the gaseous nitric oxide (NO) delivery device 1, and the treated blood is then returned to either a patient 2 or a stored blood source 3, through line 100. The treated blood should not contain toxic levels of nitric oxide when it enters the patient 2 or the stored blood source 3.

A reason it should not contain such nitric oxide of predetermined quantities is to avoid the formation of methemoglobin. If sufficient quantities of methemoglobin—a brownish-red form of hemoglobin that occurs when hemoglobin is oxidized either during decomposition of the blood or by the action of various oxidizing drugs or toxic agents; It contains iron in the ferric state and cannot function as an oxygen carrier—are formed, it could result in the death of the patient. As set forth below, the extracorporeal blood is exposed to nitric oxide for an extended time frame, a high concentration or a modification of high concentration and extended time frame. In any case, when blood is exposed to such levels of nitric oxide that can decrease and/or reduce pathogens in the blood, the blood has the ability to form methemoglobin. To counteract this possible formation of methemoglobin in an extracorporeal setting, the present invention incorporates an optional free-radical scavenger unit 66 prior to the blood entering the patient 2 or the storage source 3 and post the addition of nitric oxide.

The free-radical scavenger unit 66 can contain any conventional free-radical scavenger. An example of such a conventional free-radical scavenger includes and is not limited to citric acid. In any case, the free-radical scavenger is exposed to the treated blood and cleanses the blood of residual nitric oxide, obviously, the nitric oxide is not entirely removed from the blood but it is sufficiently removed that it should not pose an obstacle to the patient's health.

In FIG. 9, the nitric oxide (NO) gas source 7 is a pressurized cylinder containing nitric oxide (NO) gas. While the use of a pressurized cylinder is the preferable method of storing the nitric oxide (NO) containing gas source 7, other storage and delivery means, such as a dedicated feed line can also be used. Typically the nitric oxide (NO) gas source 7 is a mixture of N₂ and NO. While N₂ is typically used to dilute the concentration of NO within the pressurized cylinder, any inert gas can also be used.

When the NO gas source 7 is stored in a pressurized cylinder, it is preferable that the concentration of NO in the pressurized cylinder fall within the range of about 800 ppm to about 1200 ppm. Commercial nitric oxide manufacturers typically produce nitric oxide mixtures for medical use at around the 1000 ppm range. Extremely high concentrations of NO are undesirable because accidental leakage of No gas is more hazardous, and high partial pressures of NO tends to cause the spontaneous degradation of NO into nitrogen. Pressurized cylinders containing low concentrations of NO (i.e., less than 100 ppm NO) can also be used in accordance the device and method disclosed herein. Of course, the lower the concentration of NO used, the more often the pressurized cylinders will need replacement.

FIG. 9 also shows source of diluent gas 11 as part of the NO delivery device 1 that is used to dilute the concentration of nitric oxide (NO) for delivery to the gaseous nitric oxide (NO) delivery device 1 through line 13. The source of diluent gas 11 can contain N₂, O₂, air, an inert gas, or a mixture of these gases. It is preferable to use a gas such as N₂ or an inert gas to dilute the NO concentration since these gases will not oxidize the nitric oxide (NO) into NO₂, as would O₂ or air. The source of diluent gas 11 is shown as being stored within a pressurized cylinder. While the use of a pressurized cylinder is shown in FIG. 9 as the means for storing the source of diluent gas 11, other storage and delivery means, such as a dedicated feed line can also be used. The nitric oxide (NO) gas from the nitric oxide (NO) gas source 7 and the diluent gas from the diluent gas source 11 preferably pass through flow control valve/pressure regulators 8,120, to reduce the pressure of gas that is admitted to the gaseous nitric oxide (NO) delivery device 1.

The respective gas streams pass via tubing 9, 13, to an optional gas blender 15. The gas blender 15 mixes the nitric oxide (NO) gas and the diluent gas to produce a nitric oxide (NO)-containing gas that has a reduced concentration of nitric oxide (NO). Preferably, the nitric oxide (NO)-containing gas that is output from the gas blender 15 has a concentration that is less than about 200 ppm. Even more preferably, the concentration of nitric oxide (NO)-containing gas that is output from the gas blender 15 is less than about 100 ppm. The nitric oxide (NO)-containing gas that is output from the gas blender 15 travels via tubing 160 to a flow control valve 17. The flow control valve 17 can include, for example, a proportional control valve that opens (or closes) in a progressively increasing (or decreasing if closing) manner. As another example, the flow control valve 17 can include a mass flow controller. The flow control valve 17 controls the flow rate of the nitric oxide (No)-containing gas that is input to the gaseous nitric oxide (NO) delivery device 1. The nitric oxide (NO)-containing gas leaves the flow control valve 17 via flexible tubing 180. The flexible tubing 180 attaches to an inlet of the gaseous nitric oxide (NO) delivery device 1. The inlet for 1 might include an optional one-way valve that prevents the backflow of gas.

In one preferred embodiment of the invention, the gaseous nitric oxide (NO) delivery device unit 1 includes an NO sensor 140 that measures the concentration of nitric oxide (NO) in the treated blood or fluid stream. The nitric oxide (NO) sensor 140 and a nitric dioxide sensor (141 can be within the sensor 140 or a separate unit) preferably report the concentrations of NO and NO₂ to a controller within the gaseous nitric oxide (NO) delivery device 1, for source gas flow control and alarm. The sensors, 140, 141 can be chemilluminesence-type, electrochemical cell-type, or spectrophotomentric type sensors.

In a similar embodiment, the present invention takes the nitric oxide gas composition in line 18 and directs the nitric oxide gas composition into a patient's breathing orifice, like a nose and/or mouth. The delivery device can be a conventional gas distribution system 199, including and not limited to a conventional gas mask, conventional plastic tubing—like a nasal canula—, or through a conventional ventilator.

FIG. 10 illustrates a block diagram representation of the device 220, which can be an alternative version of item 17. The device 220 has a power source 320 that provides sufficient voltage and charge to properly operate the device 220. The device 220 also has a main microprocessor 240 that controls the operation of a solenoid valve 264, also within the device 220. The solenoid valve 264 operates in conjunction with operating parameters that are entered via a data entry keypad 202 and the input from a pressure sensor 280.

The operating parameters and the operating status of the device 220 are displayed on an LCD display 210.

The device 220 has a pressure regulator 266. The pressure regulator 266 reduces the pressure of the nitric oxide to less than 100 psi so it can be administered to the patient 2 without damaging the patient's organs, in particular the lungs, from too much pressure.

Calibrating the flow through the solenoid valve 264 is obtained by selecting the pressure of the pressure regulator 266 and controlling the time that the solenoid valve 264 is open. Thereby, the valve 264 allows a precise amount of nitric oxide gas composition to be delivered through the gas delivery line 18, which delivers the nitric oxide to the patient's breathing orifice(s). The pressure sensor 280 is designed to detect a drop in pressure in the gas delivery line 18, when the patient initiates a breath. This pressure drop signals the main processor 240 to open the solenoid valve 264 for a pre-programmed period of time. Among the parameters that are programmed into the device are: Total Breaths, Start Delay, Pulse Time, Pulse Delay, and Re-trigger Lock.

The programmable parameters are defined as follows:

Total Breaths: This parameter is the number of breaths programmed into a run of the device 220. Each time a breath is detected as identified above, a pulse of nitric oxide gas composition is injected into the breath of patient 2. Breaths that occur during a locked out time of the predetermined time frame are not counted as breaths. After the programmed number of breaths are counted, the program stops automatically and nitric oxide gas composition is no longer injected into any breaths of the patient. This number can be set anywhere from 0 to unlimited number of breaths. If the number is set at 0 then the auto shutoff is disabled and breaths will be injected with nitric oxide until the user stops the device.

Start Delay: This parameter is the programmed delay time in minutes that the user can set. The injection of nitric oxide gas composition into each breath will begin automatically after “Start Delay” minutes. It will then continue for the number of Total Breaths and then the device 220 stops automatically.

Pulse Time: This parameter is the length of time that the solenoid valve 264 will open for delivery of nitric oxide gas composition. The resolution is 0.1 seconds and the range is 0.1 sec to 0.9 seconds. If the regulator is set at 50 psi then each second of the solenoid valve 264 opening 31 cc of nitric oxide gas composition. If the regulator pressure is set at 30 psi then each 0.1 sec solenoid valve 264 opening represents 21 cc of nitric oxide gas composition. For example, if the regulator is set at 50 psi and the pulse time is set at 0.3 seconds then each detected breath will be injected with a pulse of 0.3 seconds or about 90 cc of nitric oxide gas composition.

Pulse delay: This parameter is the length of time that the machine waits after detecting the beginning of a breath before opening the solenoid valve 264 to inject a pulse of nitric oxide gas composition. This allows the user to control the position of the bolus of nitric oxide gas composition in the breath. For example, if the user sets the solenoid valve 264 at 0.4 seconds, then 0.4 seconds after the beginning of the breath is detected the solenoid valve 264 will open to inject the nitric oxide gas composition pulse.

Retrigger Lock: This parameter is the total time that the machine will ignore new breaths beginning at the detection of a new breath. If this parameter is set at 4.5 seconds then the device 220 will wait, after detecting a breath, for 4.5 seconds before recognizing a new breath. Full or half breaths that are initiated by the patient during this lockout time will not be counted and no nitric oxide gas composition will be injected. If the breath is initiated before the lockout expires and the patient is still inhaling when the lockout expires then it will be recognized as a new breath and it will be counted and injected with nitric oxide gas composition.

The data entry keypad 202 contains five active button switches defined as follows:

START/PULSE KEY: This key is used to start a run. The user is required to confirm the start by pressing an UP key or to cancel by pressing a DOWN key. When a run is in progress, pressing this key will cause the run to pause. The run is then resumed by pressing the UP key or stopping the run by pressing the DOWN key.

UP key: This key is used to confirm the start of the run, to resume a paused run and also to increment valve changes.

DOWN key: This key is used to cancel a started run, end a paused run and also to decrement valve changes.

NEXT key: This key is used to switch screen pages on the LCD display.

PURGE key: This key is used to open the solenoid valve 264 for two seconds to purge the line. This key is not active during a run. The LCD display can display at least four screen pages, defined as follows:

Each screen page displays a status line. The status variations include NOT RUNNING, WAITING, RUNNING, PAUSED, PURGING and START Pressed.

The main screen page has a row of asterisks on the top line. This is the only screen available when the KEY switch is in the locked position. This screen displays the total breaths detected and also the total breaths that will cause the run to stop.

The second page shows two valves. The first is the START DELAY valve. When the screen first appears the blinking cursor shows the value, which can be changed by pressing either the UP or DOWN key. By pressing the NEXT key switch the cursor to the second value on the screen is TOTAL BREATHS.

The third page allows the user to change the PULSE DELAY and the PULSE TIME.

The fourth page allows the user to change the RETRIGGER LOCK.

In any case, this embodiment of the invention allows the nitric oxide gas composition to be injected into a patient's lung, preferably when the patient is inhaling, of a sufficient quantity that nitric oxide is capable of penetrating both the epithelial and capillary basement membranes to allow the nitric oxide to contact the numerous blood cells to reduce pathogens in the blood system and throughout the body.

Alternatively, this latest method can provide the nitric oxide gas continuously, just not when the patient 2 inhales. In addition, this embodiment can be used not with high concentrations of nitric oxide, but with extended durations of the nitric oxide. This embodiment allows the patient to receive low concentrations of nitric oxide over an extended time frame to reduce the pathogens within the blood stream of the patient 2. The present embodiment, in contrast to the extracorporeal embodiment, does not need to control the formation of methemoglobin due to the extended duration and low concentration of the nitric oxide which has a decreased chance of forming such methemoglobin. Values and other embodiments thereof of providing nitric oxide to a patient's lungs (and by default) blood can be found in commonly assigned international application no. PCT/CA99/01123, which is hereby incorporated by reference herein.

It is appreciated that various modifications to the inventive concepts described herein may be apparent to those of ordinary skill in the art without departing from the scope of the present invention as defined by the herein appended claims.

We claim:

1. A method of reducing pathogens in a mammal's blood stream by exposing the blood in an extracorporeal fluid with nitric oxide, comprising:
(a) providing an extracorporeal blood circuit comprising an inlet line adapted to receive blood from a patient or a blood source, an outlet line adapted to return blood to the patient and/or the blood source, a fluid circuit for fluid communication between the inlet and the outlet line, and at least one pump acting on the fluid circuit to circulate blood therethrough and out the outlet line,
(b) circulating the blood through the extracorporeal blood circuit, and
(c) exposing the blood in the circuit with nitric oxide gas in a concentration sufficient to reduce pathogenic content in the blood.

2. The method of claim 1 further comprising including in the circuit a blood treatment component, treating blood with the component, and at least upstream of the component contacting blood in a portion of said circuit with nitric oxide gas in concentration sufficient to reduce pathogenic content in the blood.

3. The method of claim 2 further comprising selecting said component from the group consisting of a dialysis component, an organ perfusion component, a heat exchange component, an oxygenation component, or a combination thereof.

4. A method of reducing pathogens in blood in an extracorporeal fluid circuit of a cardiopulmonary bypass apparatus, comprising:
providing a cardiopulmonary bypass circuit that includes an inlet line adapted to receive blood from a patient or a blood source and an outlet line adapted to return blood to the patient and/or blood source, a reservoir connected to the inlet line for accumulation of blood received from the patient, an oxygenator, a fluid interconnection circuit for fluid communication between the reservoir and the oxygenator and between the oxygenator and the outlet line, and at least one pump acting on the fluid interconnection circuit to withdraw blood from the reservoir and circulate it through the oxygenator and out the outlet line, and
exposing the blood in a portion of the cardiopulmonary bypass circuit with nitric oxide gas in concentration sufficient to reduce pathogens in the blood.

5. The method of claim 4 further comprising:
monitoring the rate of flow of blood through the cardiopulmonary bypass circuit,
introducing nitric oxide gas into the circuit, and
controlling the pressure and rate of flow of gas introduced into the circuit in relation to the flow of blood through the circuit to maintain the concentration of nitric oxide within a desired range sufficient to reduce pathogens of the blood.

6. The method of claim 4 comprising introducing nitric oxide into a blood accumulator reservoir receiving blood from the patient for exposing with the blood.

7. The method of claim 4 comprising
locating a semipermeable membrane selectively permeable to nitric oxide gas and impermeable to nitrogen gas in a portion of said fluid interconnection circuit distally proximate the reservoir in a longitudinal disposition adapted to allow contact of an outside of the membrane with blood flowing through the fluid interconnection circuit portion, and
delivering nitric oxide gas to the inside of the membrane under pressure sufficient to drive the nitric oxide across the membrane for contact with blood on the outside of the membrane within a desired concentration range sufficient to reduce pathogens in the blood.

8. The method of claim 7 further comprising
providing the membrane in tubular form having an inlet and outlet and in coaxial disposition within the fluid interconnection circuit portion,
delivering the nitric oxide with a nitrogen carrier gas through the inlet and removing gas through the outlet sufficient to maintain the pressure and rate of flow, and
scavenging any nitric oxide present in the gas removed through the membrane outlet.

9. The method of claim 1 wherein the pathogens are septicemia and/or bacteremia.

10. The method of claim 4 wherein the pathogens are septicemia and/or bacteremia.

11. The method of claim 1 wherein the nitric oxide gas is mixed with other gases.

12. The method of claim 4 wherein the nitric oxide gas is mixed with other gases.

13. The method of claim 1 further providing a free-radical scavenger unit that exposes the blood to free-radical scavengers after the blood is exposed to the nitric oxide.

14. The method of claim 4 further providing a free-radical scavenger unit that exposes the blood to free-radical scavengers after the blood is exposed to the nitric oxide.

15. An extracorporeal blood circuit comprising an inlet line adapted to receive blood from a patient or a blood source, an outlet line adapted to return blood to the patient and/or the blood source, a fluid circuit for fluid communication between the inlet and the outlet line, and at least one pump acting on the fluid circuit to circulate blood therethrough and out the outlet line,
a nitric oxide unit that exposes the blood in the circuit with nitric oxide gas in a concentration sufficient to reduce pathogenic content in the blood; and
a free radical scavenger unit that exposes the blood in the circuit and after being exposed to nitric oxide, with a free-radical scavenger in a concentration sufficient to reduce the nitric oxide content in the blood.

16. A nitric oxide gas dispenser for mammals, comprising:
a component that provides nitric oxide gas and or aerosolized composition at a desired pressure and concentration, and a delivery system that provides the nitric oxide at the desired pressure to the mammal;
a valve mechanism that controls the flow of the nitric oxide so the mammal's lungs receive a predetermined amount of nitric oxide and the nitric oxide is of sufficient quantity that it is able to penetrate through the mammal's lungs to contact the mammal's blood cells to reduce the pathogens in the mammal's blood and not form excessive amounts of methemoglobin.

17. The dispenser of claim 16 wherein the dispenser has a pressure sensor positioned along the delivery system and determines when the mammal is taking a breadth;
if the pressure sensor determines the mammal is taking a breadth, the pressure sensor transmits a breadth signal to a microprocessor, the microprocessor then determines if the mammal is within a prescribed time frame for the mammal to be administered nitric oxide;
if the microprocessor determines the mammal is within the prescribed time frame, the microprocessor transmits an open signal to the valve mechanism to release the predetermined amount of nitric oxide to the mammal to reduce pathogens in the blood system.

18. The dispenser of claim 16 wherein the nitric oxide is transmitted into at least one nostril of the mammal, or the mouth of the mammal, or through a ventilator.

19. The dispenser of claim 16 wherein the pathogens are septicemia and/or bacteremia.

20. The dispenser of claim 16 wherein the nitric oxide is mixed with other gases.

21. A method of reducing pathogens in a mammal's blood stream by exposing the blood that receives oxygen from the lungs with nitric oxide, comprising:
(a) administering nitric oxide from a nitric oxide dispenser unit to a mammal through a nasal canula, a mask or a ventilator circuit for a mammal breathing or on ventilator like support;
(b) exposing the blood in contact with the patient's lungs with nitric oxide in a concentration sufficient to reduce pathogenic content in the blood.

22. The method of claim 21 wherein the pathogens are septicemia and/or bacteremia.

23. The method of claim 21 wherein the nitric oxide is a gas.

24. The method of claim 21 wherein the nitric oxide is delivered through an aerosolized donor compounds.

25. The method of claim 21 wherein the nitric oxide is mixed with other gases.
